(19) United States
(12) Patent Application Publication
(10) Pub. No.: US 2014/0257390 A1
(43) Pub. Date: Sep. 11, 2014

(54) Osseointegrative Spinal Fixation Implants

(71) Applicant: Kenneth M Little, Boise, ID (US)

(72) Inventor: Kenneth M Little, Boise, ID (US)

(21) Appl. No.: 14/199,999

(22) Filed: Mar. 6, 2014

Related U.S. Application Data
(60) Provisional application No. 61/773,937, filed on Mar. 7, 2013.
(60) Provisional application No. 61/776,375, filed on Mar. 11, 2013.

(51) Int. Cl. A61B 17/70; A61L 27/04

(52) U.S. Cl. CPC A61B 17/7058; A61B 17/7062; A61L 27/04
USPC 606/246

(57) ABSTRACT

The present invention describes implant systems, devices, methods and surgical techniques for spinal fixation that incorporate an osseointegrative bone-implant interface that functionally provides both the short term stability of fixation and the long term stability of fusion. The various embodiments described herein can utilize novel screw and anchoring device designs or can serve as a supplement to existing spinal fixation systems.

### CROSS-REFERENCE TO RELATED APPLICATIONS

This application claims priority from U.S. Provisional Patent Application No. 61/773,937, filed Mar. 7, 2013, entitled “Osseointegrative Spinal Fixation Implants” and U.S. Provisional Patent Application No. 61/776,375, filed Mar. 11, 2013, entitled “Osseointegrative Spinal Fixation Implants,” the contents of which are hereby incorporated herein by reference.

### TECHNICAL FIELD

The disclosure generally relates to the field of spinal surgery implants. More particularly, the present invention discloses devices, methods, systems and surgical procedures for spine surgery, including system in the fields of posterior and posterolateral cervical, thoracic, lumbar, and sacral hardware fixation devices and natural or synthetic osseointegrative materials.

### BACKGROUND OF THE INVENTION

Conventional methods of posterior and posterolateral cervical, thoracic, lumbar, and sacral spinal fusion surgery largely rely upon both fixation and fusion. Spinal fixation involves the use of metal and/or polymer implants to provide immediate stability after spine surgery, and such systems often seek to “fix” mobile spinal segments to promote an ultimate goal of a more durable stability typically afforded by bone growth between the native anatomy and surgical implant (i.e., fusion). Where vertebral bodies or other bony anatomical structures of the spine are spanned by an arthrodesis or induced bony mass, such “fusion” of spinal segments provides long-term stability. In general, a surgeon will attempt to induce fusion by placement of autologous or cadaveric bone graft, with or without synthetic bone graft substitutes and osteoinductive agents.

In general, relying upon implants for spinal fixation as well as graft material to achieve long-term stability (fusion) inflates the cost of surgery. Autologous bone graft is the “gold standard” to achieve spinal fusion, but the collection and use of such materials can be painful and subject patients to additional risk for a variety of reasons. While the use of allograft and/or bone graft substitutes may ameliorate some of those patient risks, these materials are generally thought of as not as reliable as autologous bone graft, and they can also inflate the cost of surgery as well as subject patients to additional risks.

Osseointegrative materials are materials having a porous surface into which bone forming cells can migrate. In the case of an implant having a surface that incorporates osseointegrative materials, such bone ingrowth can allow for a much more rigid bond between native bone and various portions of a load-bearing artificial implant. Various materials and methods of creating implants with porous, osseointegrative surfaces have been created and used in orthopedic and dental surgery. Osseointegrative materials have also been incorporated into spinal fusion implants, including porous metallic plating system implants used in anterior inter-vertebral body fusion of the cervical spine. Such designs can promote the effective “fusion” of treated spinal segments, where the patient's bone can grow into the porous implant surface and secure the device to the relevant bone, while the structural stability between the bones is provided by the solid body of the implant itself. However, a number of disadvantages are attendant with such plating systems, including that these implants are specifically designed and intended for anterior surgery (i.e., placed on the anterior faces of adjacent vertebral bodies) and the implantation of such devices typically presents a greater technical challenge to place (as compared to other surgical approach directions), can often increase the risks of surgery, and such systems often rely upon the use of additional implants for fixation.

In posterior spinal surgery, pedicle screws are a commonly used and well accepted type of fixation implant. Pedicle screw fixation of implants typically relies upon mechanical fixation of screw threads or flutes within the bony structure(s) of the pedicle and vertebral body for immediate fixation of implant components. In some cases, pedicle screws have included an osseointegrative hydroxyapatite coating (to desirably promote fusion between the screw body and the surrounding support bone), which might be used in some osteoporotic patients, but bone graft material is still typically required to achieve fusion between the treated vertebral bodies.

### BRIEF SUMMARY OF THE INVENTION

The present invention includes the realization of a need for a spinal implant system for fusion and arthrodesis surgeries that promotes and enables the formation of an arthrodesis along an osseointegrative path provided by the implant system, with the path spanning the entire distance between the relevant supported bony anatomical features. In one embodiment, the present invention discloses a spinal implant system having an osseointegrative bone-implant interface portion and associated structural support portion that functionally provide both the short term stability of fixation and the long term stability of fusion. The system can include one or more longitudinally-extending elements that can be desirably used with a variety of pre-existing posterior spinal fixation screws (including pedicle screws) or can be used in conjunction with a novel posterior screw that incorporates a poly-axial post. In some applications the longitudinal device could be homogenous, which could include a longitudinally-extending element having at least one cross-sectional portion formed of only porous or trabecular metal, while in other more preferred applications the longitudinally-extending element could comprise a composite type and/or density of materials, including designs incorporating osseointegrative materials as well as other materials, such as polymers or higher density metals, to provide greater strength and rigidity to the construct.

In one preferred embodiment, a spinal implant system can include an elongated member having a central region of substantially solid, load bearing material such as titanium and/or cobalt chrome, with a surrounding region of osseointegrative material such as porous metal or other artificial and/or natural materials. In this embodiment, both the central region and the osseointegrative region of the member are connected to the bony anatomy via a fixation device, such as pedicle screws of other anchoring devices known in the art, with at least a portion of the osseointegrative region directly in intimate contact with bony structures of the treated anatomy.

### BRIEF DESCRIPTION OF THE SEVERAL VIEWS OF THE DRAWINGS

FIG. 1 is a top plan view of one embodiment of a composite osseointegrative member constructed in accordance with various teachings of the present invention;

FIG. 2 is a cross-sectional view of the member of FIG. 1, taken along line AA-AA;

FIG. 3 is a partial perspective cross-sectional view of the member of FIG. 1, taken along line BB-BB;

FIG. 4 is a exploded cross-sectional view of a member and associated anchoring screw, demonstrating one method of implanting the device in a patient's spine;

FIG. 5 is a posterior plan view of the member of FIG. 1, fixated to a lumbar spine;

FIG. 6 is a lateral plan view of the member and lumbar spine of FIG. 5;

FIG. 7 is a top plan axial view of the member and lumbar spine of FIG. 5;

FIG. 8 is a posterior plan view of the member of FIG. 1, fixated to a thoracic spine;

FIG. 9 is a lateral plan view of the member and thoracic spine of FIG. 8;

FIG. 10 is a top plan axial view of the member and thoracic spine of FIG. 8;

FIG. 11 is a top plan axial view of the member of FIG. 1, fixated to a cervical spine;

FIG. 12 is a posterior plan view of the member and cervical spine of FIG. 11;

FIG. 13 is a lateral plan view of the member and cervical spine of FIG. 11;

FIG. 14 is a side view of one alternative embodiment of a composite osseointegrative member constructed in accordance with various teachings of the present invention;

FIG. 15 is a top plan axial view of a lumbar vertebra showing a standard pedicle screw placement;

FIG. 16 are exploded and perspective views of a of standard pedicle screws and connecting rod system;

FIG. 17 is a perspective view of the standard pedicle screw and connecting rod system of FIG. 16;

FIG. 18 is a perspective view of another alternative embodiment of a composite osseointegrative member constructed in accordance with various teachings of the present invention;

FIG. 19 is a perspective view of the composite osseointegrative member assembled to the pedicle screw and rod assembly of FIG. 17;

FIG. 20 is a perspective view of the composite osseointegrative member and pedicle screw and rod assembly being assembled;

FIG. 21 is a perspective view of a fully assembled composite osseointegrative member and pedicle screw and rod assembly;

FIG. 22 is a top plan axial view of the fully assembled composite osseointegrative member and pedicle screw and rod assembly implanted into a lumbar vertebrae.

FIG. 23 is a perspective view of another alternative embodiment of a composite osseointegrative member constructed in accordance with various teachings of the present invention with an exploded perspective view of a standard pedicle screw and connecting rod system;

FIG. 24 is a perspective view of the composite osseointegrative member of FIG. 23 assembled to the pedicle screws and rod, with an axial with an end-on cross-sectional view of the assembly;

FIG. 25 is a perspective view of the composite osseointegrative member of FIG. 23 assembled to the pedicle screws with a top plan axial cross-sectional view of the assembly;

FIG. 26 are perspective views of various additional embodiments of composite osseointegrative members constructed in accordance with various teachings of the present invention;

FIG. 27 are perspective views of various additional embodiments of composite osseointegrative members constructed in accordance with various teachings of the present invention;

FIG. 28 is a perspective view of another alternative embodiment of a composite osseointegrative member constructed in accordance with various teachings of the present invention for use with an exemplary pedicle screw design from FIG. 27;

FIG. 29 is a perspective view of the composite osseointegrative member of FIG. 28 assembled to the pedicle screws, with an axial with an end-on cross-sectional view of the assembly; and

FIG. 30 is a perspective view of the composite osseointegrative member of FIG. 28 assembled to the pedicle screw of FIG. 27 with a top plan axial cross-sectional view of the assembly.

### DETAILED DESCRIPTION OF THE INVENTION

The disclosures of the various embodiments described herein are provided with sufficient specificity to meet statutory requirements, but these descriptions are not necessarily intended to limit the scope of the claims. The claimed subject matter may be embodied in a wide variety of other ways, may include different steps or elements, and may be used in conjunction with other technologies, including past, present and/or future developments. The descriptions provided herein should not be interpreted as implying any particular order or arrangement among or between various steps or elements except when the order of individual steps or arrangement of elements is explicitly described.

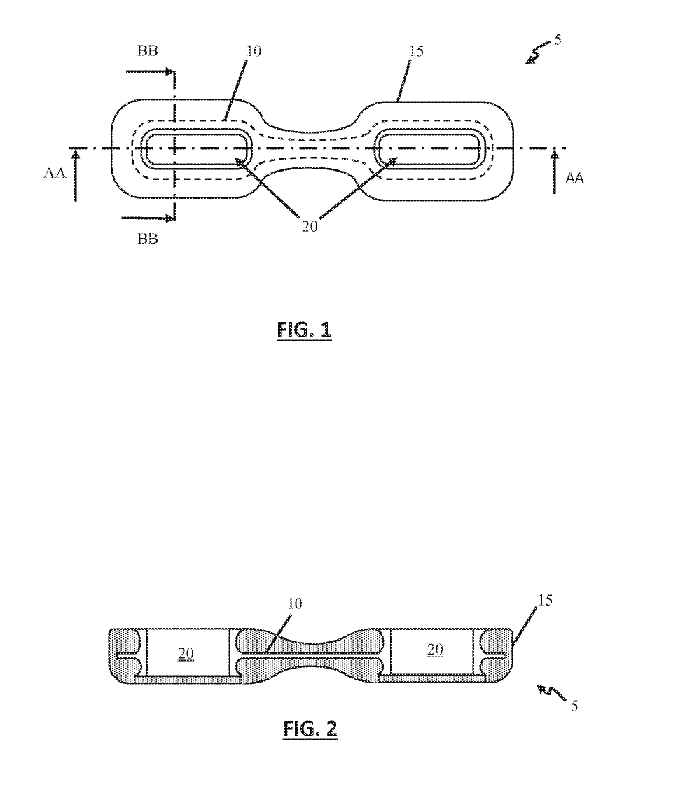

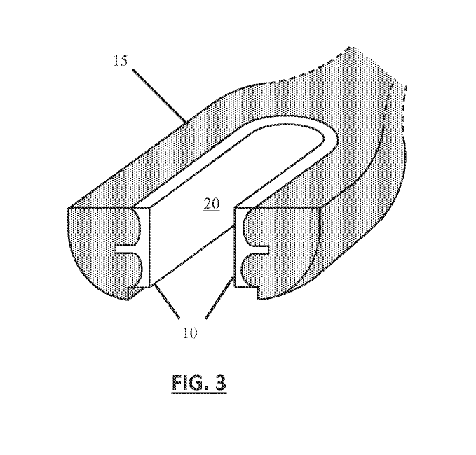

Referring now to the invention in more specific detail, FIG. 1 through FIG. 3 show one exemplary embodiment of the invention, constructed in accordance with various teachings provided herein. The device as shown is an elongated and/or semi-cylindrical plate, rod or member 5 formed as a composite structure, the member comprising a central core 10 (shown in broken line) of substantially solid metal or polymer that is forms a significant portion of the structure's initial strength and flexibility, with at least a portion of the core surrounded by an outer shell 15 of porous, osseointegrative material (such as, for example, trabecular or porous metal or other substrate with a polymer-coated surface, as well as other osteoconductive and/or osteoinductive materials and/or coatings). Desirably, at least a portion of the outer shell 15 will extend substantially the entire length of the member, although various other designs are contemplated herein. As will be described later, the outer shell 15 will desirably contact and have a direct interface with bone of the targeted anatomy, while the central core 10 will primarily interface with various instrumentation used to secure the member to the underlying vertebrae.

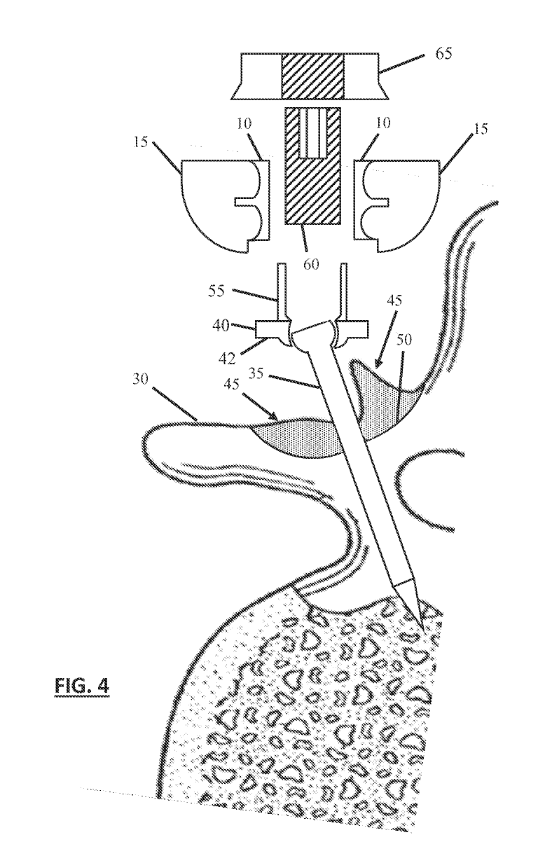

The member 5 also includes slots 20, which in the exemplary embodiment include a pair of slots 20 disposed within the central core (and also within the shell 15) and extending substantially along a longitudinal axis of the member 5. In this embodiment, the longitudinal of width of the slots desirably accommodates an outer diameter of a fixation screw head or other fixation feature (see FIG. 4) while the length of the slots 20 desirably accommodates adjustment and/or adjustability of the fixation screw(s) which is anchored to the vertebral body(ies). This slot length desirably allows for variations in distance between vertebral bodies from patient to patient. That being said, the device can be manufactured in various sizes to accommodate various ranges of distance as well as in multi-level designs to accommodate multi segment fixation, if desired.

FIG. 4 shows an exploded view of one exemplary method for securing the member 5 to a spinal bone such as a vertebrae 30, which desirably allows the device to firmly contact the underlying bone approximate to each point of fixation. In this figure, a cross-sectional view of member 5 (taken along line BB-BB of FIG. 1) is shown to demonstrate one exemplary mechanism of connection with an associated fixation device, which in this example is a transpedicular screw shank 35 having a poly-axial head 40. The figure also depicts a partial plan view of the vertebral body 30, with a desired zone of implantation 45 for the member 5, which in this embodiment of the invention within the lumbar spine can be positioned at the junction between a facet joint and a transverse process of the vertebral body 30.

Desirably, during the surgical implantation procedure the surface bone where the device will be applied is decorticated, creating a “bleeding bone” boundary 50 which can promote cellular migration into the implant's osseointegrative surface. As best seen in FIG. 4, the “bleeding bone” boundary 50 can be formed in a curved or cylindrical path, desirably in a shape that corresponds to the outer shell 15 of the member 5. Depending upon the desires of the surgeon, the boundary 50 could be prepared before, during and/or after implantation of the fixation device(s), although in many cases it may be advantageous to prepare the boundary 50 in each vertebrae after the positioning and/or orientation of the appropriate fixation device (or pair of fixation devices in adjacent vertebral bodies, for example) has been accomplished.

In one exemplary embodiment of a surgical procedure using the various devices and systems described herein, a transpedicular screw 35 can be first placed into the vertebral body 30 using standard surgical approach and placement operative techniques. The transpedicular screw 35, as shown in FIG. 4, is not depicted fully advanced into the vertebral body 30, and in actual application it would typically be advanced such that a face 42 of the poly-axial head 40 would desirably abut a surface of the bone 30 (i.e., a native bony surface and/or a prepared “bleeding bone” boundary surface). In the embodiment depicted, the screw assembly includes a screw shank 35 with an associated poly-axial head 40, the poly-axial head 40 including a flange 55 which desirably directly contacts various inner and abutting surfaces of the core 10 of the member 5, creating a solid metal-metal interface between the screw assembly and the member 5. In various alternative embodiments, this metal-metal interface could comprise a variety of feature, including irregular surfaces that may increase friction and/or lock the surfaces together, as well as to mitigate and/or inhibit rotation once implanted.

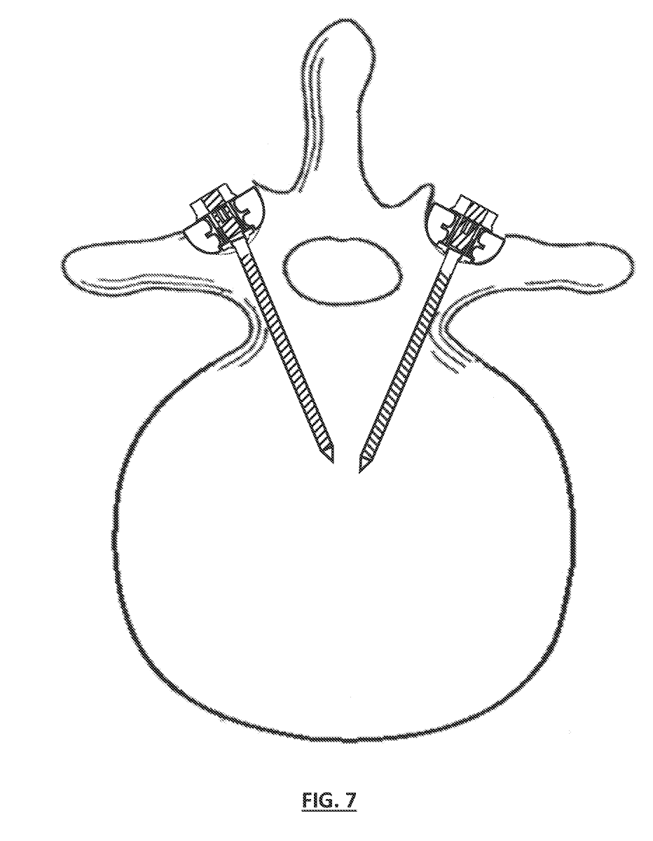

In the disclosed embodiment, the metal-metal interface between the screw assembly and the core is desirably sized and configured to be recessed within the osseointegrative shell 15 (see FIG. 7), which facilitates direct contact and opposition of the shell with the underlying bone substrate (and “bleeding bone” bed). The poly-axial head 40 can include an outer shape and diameter of flange 55 that desirably fits within the relevant device slot 20 (i.e., aligning medially-laterally to the vertebral body), with the length of the slot 20 desirably allowing for significant rostral-caudal adjustments of the member 5 relative to the various fixation screws (i.e., transpedicular screws in the depicted embodiment of this invention). Further, the poly-axial head 40 can include a threaded inner surface which accommodates an insert or set screw 60 (which may be provided integrally with the head 40, or may comprise a separate component, such as depicted herein), which in turn locks the poly-axial head 40 in a desired orientation relative to the screw 35. The head of the set screw 60 can include an inner hexagonal head 65 which facilitates advancement of the set screw 60 using a hexagonal screw driver or other appropriate surgical device.

To fully assemble the system, one or more transpedicular screw shanks 35 can be placed into the relevant vertebral bodies in a known manner, and then the member 5 can be lowered over the poly-axial head 40 and placed into intimate contact with the underlying bleeding bone boundary. The set screw 60 can then be inserted into the poly-axial head 40, and tightened to lock the poly-axial head 40 into a desired position relative to the screw shank 35 (which may also cause the flange 55 to expand to some degree, potentially wedging against inner surfaces of the core 10). The set screw 60 can also include a threaded upper portion that serves as a post upon which an outer nut 65 can be placed. Tightening the outer nut 65 can firmly secure the member 5 to the fixed poly-axial head 40.

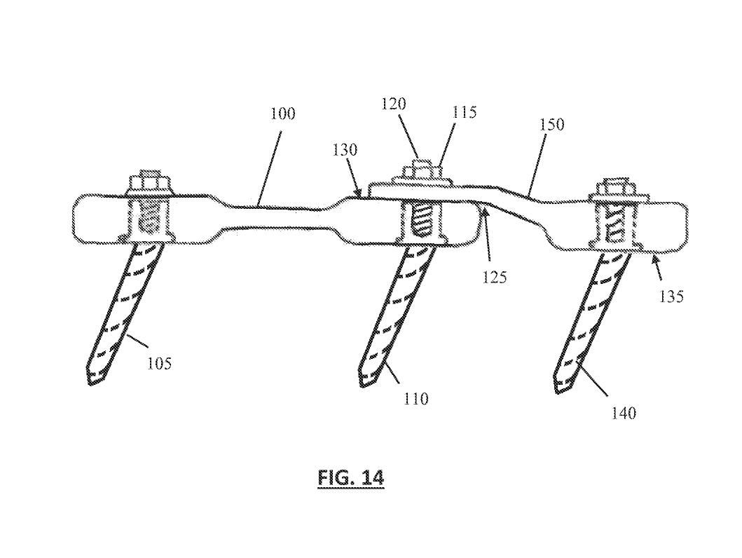

In one additional embodiment, such as shown in FIG. 14, longer set screws 60 could be used to accommodate other configurations of the various devices described herein, including the employment of a “piggyback” configuration of devices for the instrumentation of adjacent spinal segments.

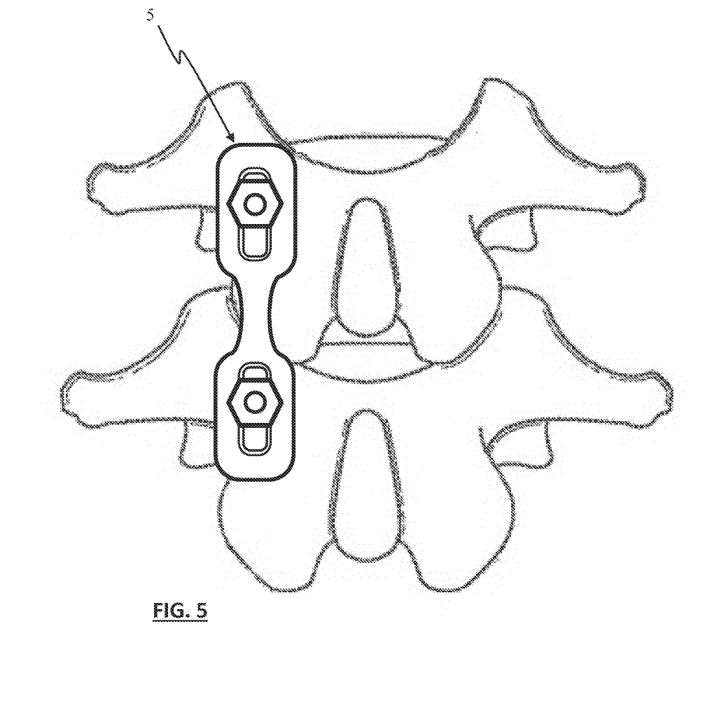

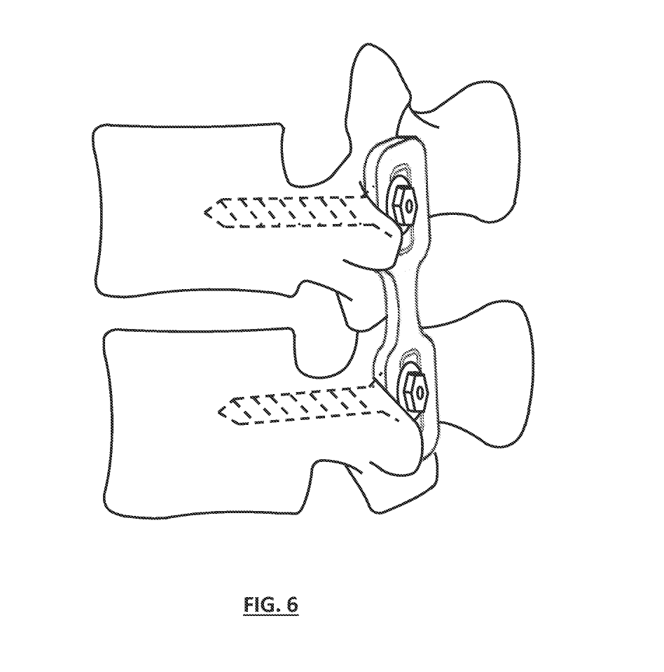

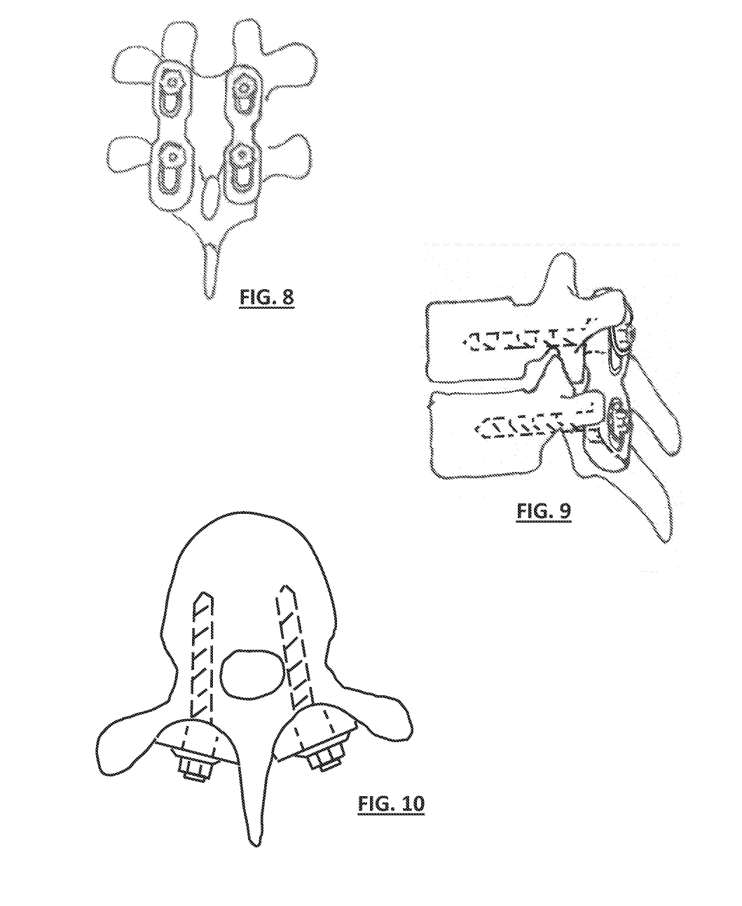

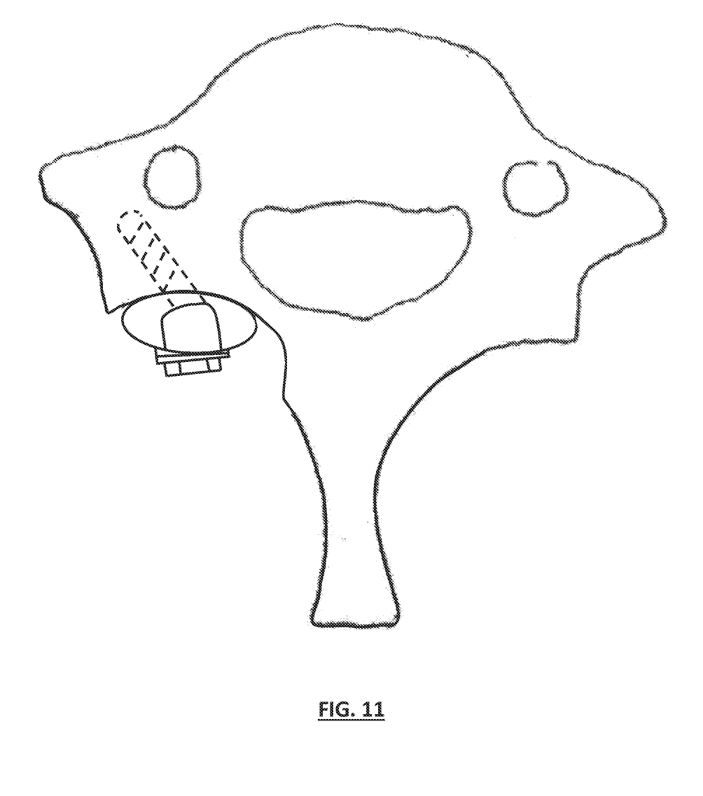

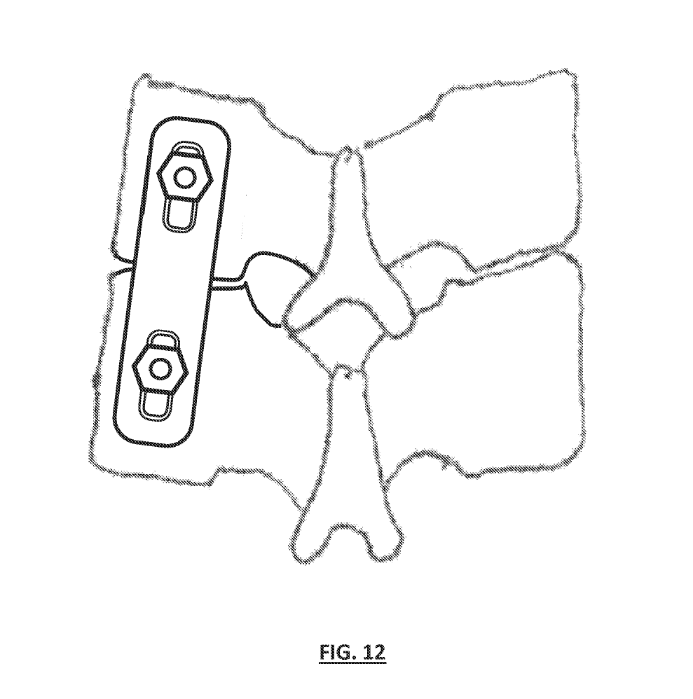

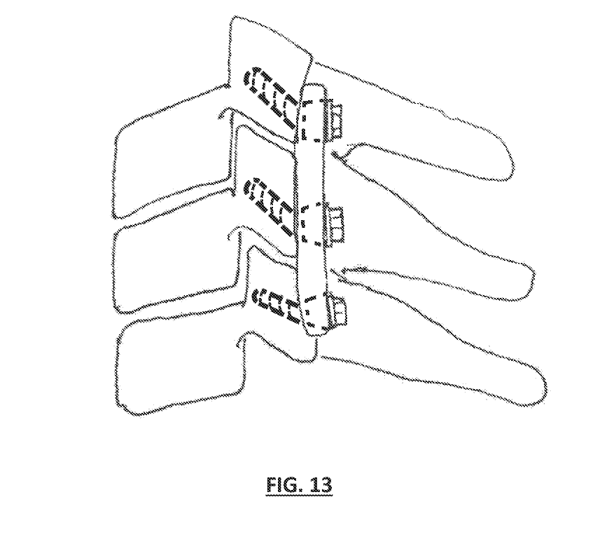

FIGS. 5 through 7 show various views of device embodiments implanted into a lumbar spine. FIGS. 8 through 10 show various views of device embodiments implanted into a thoracic spine and FIGS. 11 through 13 show various views of device embodiments implanted into a cervical spine. In the exemplary embodiment of FIG. 13, a member is depicted implanted into three vertebral bodies across two motion segments.

FIG. 14 depicts one alternative embodiment of a system particularly well suited for multi segment implantation. In this embodiment, a member 100 and associated fixation screws 105 and 110 could have been implanted at a previous surgery or at a previous point during a current surgery. However, it may at some point become necessary to add an additional member 150 to the existing construct. Rather than removing a previously osseointegrated implant (from the prior surgery) or significantly disturb the existing construct, the original outer nut 115 of a fixation screw 110 of the existing construct could be removed, and the existing set screw (not shown) loosened and replaced with a longer set screw 120, which could accommodate an alternative embodiment of an additional member 150, which can be “piggy-backed” onto the previously implanted device and tightened in place using the original outer nut 115, if desired. Desirably, this additional member 150 will be constructed in a manner similar to the member 100 previously described, which could include the incorporation of a generally solid core region substantially surrounded by a porous material. In the embodiment depicted, the additional member desirably includes a porous lower surface 125 generally facing towards (and in substantial contact with) a corresponding porous upper surface 130 of the member 100, which desirably induces bony integration of the member 100 and additional member 150 as described herein. Moreover, the additional member 150 also desirably includes a porous lower surface 135 proximate to the fixation screw 140 (or other fixation device), which desirably facilitates bony integration with the prepared bony anatomy, as previously described herein.

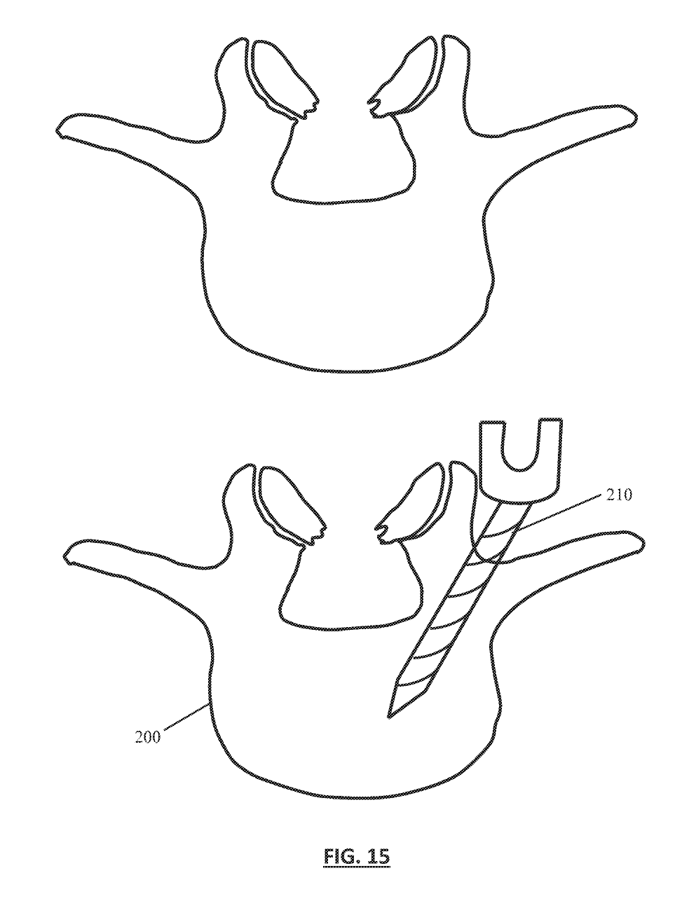

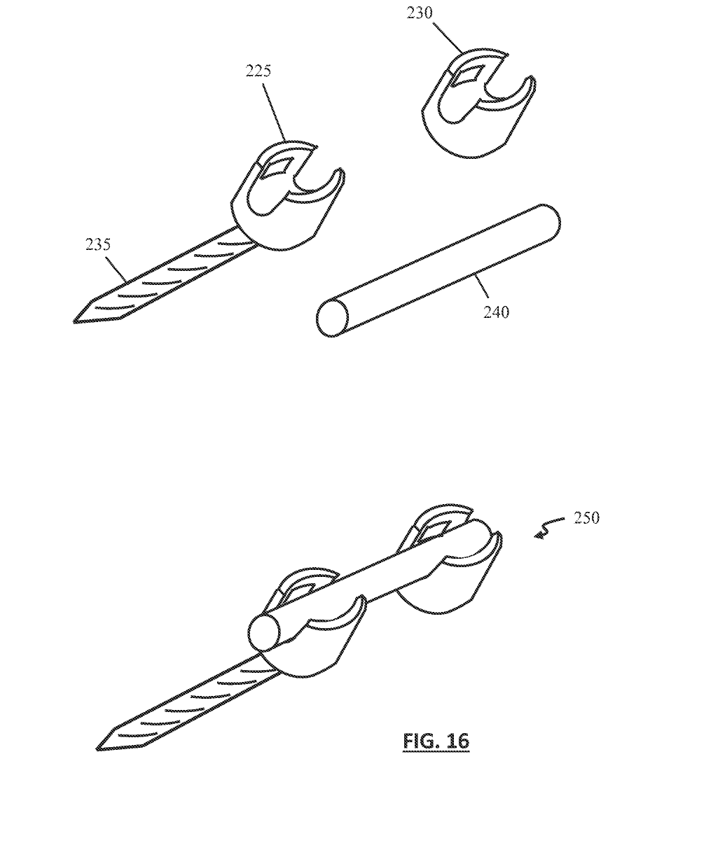

FIGS. 15-22 depict another additional embodiment of the invention, incorporating various design features that can facilitate connect of the osseointegrative member to standard posterior fixation hardware. In the example of FIGS. 15 and 16, a typical spinal construct including lumbar pedicle screws and an associated fixation rod are shown; however, it should be understood that various features of the present disclosure could be adapted to accommodate a wide variety of other posterior fixation screws (or other fixation devices, including those for anterior and/or lateral fixation) in the cervical, thoracic, lumbar, sacral, or iliac spine. FIG. 15 shows an axial view of a lumbar vertebra 200 as well as an exemplary position for a typical pedicle screw 210 placed at a typical entry point and trajectory.

FIG. 16 shows a pair of standard poly-axial pedicle screw heads 225 and 230, with slot openings oriented towards one another in a manner typical of in-situ implantation in a patient's spine. One screw head 225 is depicted with a screw shank 235 shown, while the other screw head 230 is depicted (for convenience) without a shank shown. A standard longitudinal rod segment 240 is also shown, with the assembled pedicle screw and rod assembly 250 depicted at the bottom of FIG. 4 (without set screws in this figure).

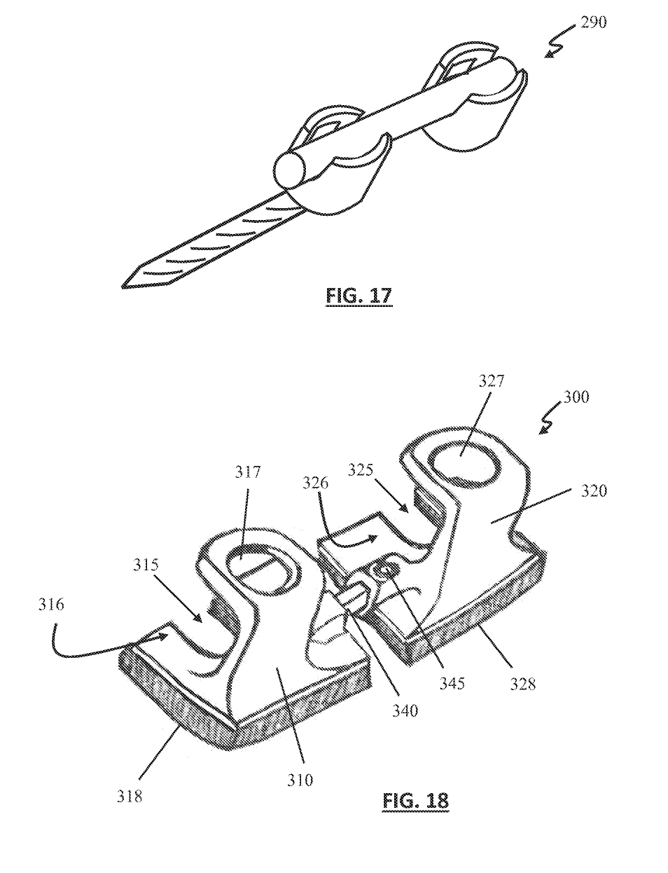

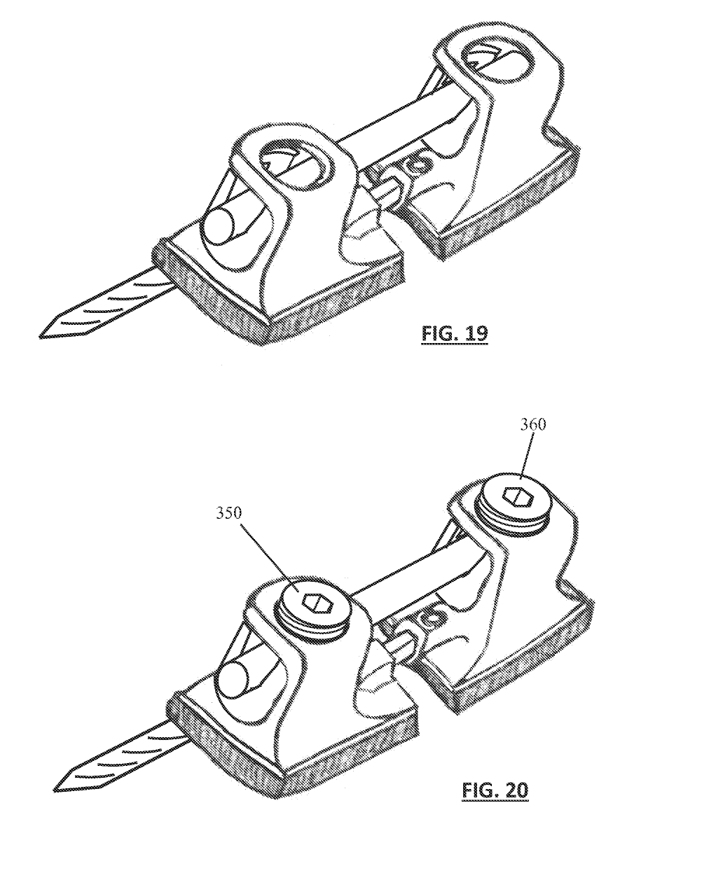

FIG. 17 shows a typical pedicle screw and rod assembly 290 before integration with an embodiment of the present invention that can be utilized to “retrofit” a standard spinal construct into a osseointegrative construct. FIG. 18 shows one embodiment of an osseointegrative construct 300 designed in accordance with various teachings of the present disclosure. The construct 300 includes a first engagement body 310 and a second engagement body 320, each of the engagement bodies including a lower slot 315 and 325 for accommodating a screw shank on the fixation elements of the rod and screw assemble 290, and each engagement body 310 and 320 further includes a clamp opening 316 and 326 for accommodating a head (i.e., mono or poly axial head) and associated fixation rod of the rod and screw assembly 290. Each engagement body 310 and 320, which can be formed from solid metallic, ceramic and/or plastic materials, desirably includes at least a lower surface portion 318 and 328 comprising an osseointegrative material (i.e., a porous or trabecular metal or polymer coating or other materials known in the art) that can directly contact an underlying bony anatomical surface, such as a “bleeding bone” surface of a vertebral body (not shown). In use, the lower surface portion will desirably facilitate the formation of a bony attachment to the osseointegrative material, while the stronger and more rigid metal (or other material) of the engagement body 310 and 320 will provide a strong interface with the pedicle screw and rod assembly 290. In the disclosed embodiment, the engagement bodies 310 and 320 can be inserted from lateral to medial in such a way that the proximal pedicle screw (just distal to the poly-axial head) slides into and engages with the clamp opening 316 (see FIG. 19). In addition, openings 317 and 327 are provided on the top of each engagement body 310 and 320, which may be used to access the top of each pedicle screw head to facilitate loosening and/or tightening of the screw and rod assembly 290 as well as advancing/withdrawing the pedicle screw and/or placing a set screw, as desired. In the disclosed embodiment, the engagement bodies 310 and 320 can be inter-connected using a small caliber rod 340 extending the bodies, which can be locked into place using a set screw 345 or other arrangement. If desired, the rod 340 could incorporate osseointegrative material (not shown) which may be provided in intimate contact with one or both of the lower surface portions, in a manner similar to those previously described herein.

FIG. 19 shows the construct 300 of FIG. 18 placed into position over the pedicle screw and rod assembly 290 of FIG. 17. In this figure it can be seen that the rod segment is shown in a typical position and/or orientation relative to the pedicle screws for a single level fusion (or possibly a skip-level fusion). In various embodiments, depending upon how the pedicle screw and rod assembly may be positioned on a patient's anatomy (as well as the condition and positioning of any intervening anatomy) the construct 300 is designed to desirably allow the rod segment to be placed before, after and/or during the placement of the engagement bodies 310 and 320 over the pedicle screw heads. Such a design can allow a surgeon to advance and/or reposition the pedicle screws after the components of the construct 300 have been placed, which can assist with achieving adequate apposition of the osseointegrative material of the lower surfaces to the decorticated vertebral bone substrate.

FIG. 20 depicts the assembled construct 300 and pedicle screw and rod assembly 290 of FIG. 19, with set screws 350 and 360 in place. The sets screws 350 and 360 may be longer than standard set screws, which are typically designed to be flush with the top of the poly-axial screw head. The set screws 350 and 360 of this embodiment of the invention may be significantly longer, if desired, and as depicted have threads exposed above the engagement bodies to allow for placement of locking screws.

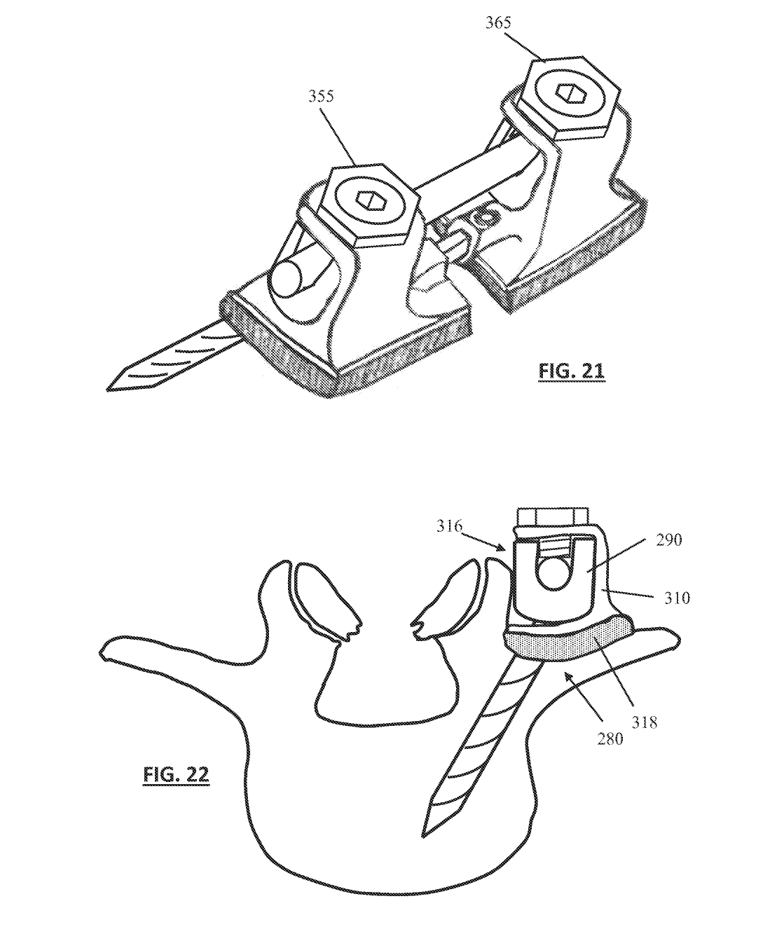

FIG. 21 depicts a pair of locking screws 355 and 365 that are positioned over the set screws 350 and 360 of FIG. 20. A single segment instrumentation construct is depicted in the figure, although multi-segment treatment is also contemplated in the present embodiment. If desired, adding additional longitudinal connections, including those similar to the small caliber rod 340 and set screw 345 arrangement of FIG. 18, could provide a useful multi-level construct.

FIG. 22 depicts an axial view of an in-situ depiction of the embodiment of FIGS. 15-21. The pedicle screw and rod construct 290 is desirably captured within the clamp opening 316 of the engagement body 310, with the osseointegrative material of the lower surface 318 apposed and in direct contact with the underlying decorticated vertebral bone 280.

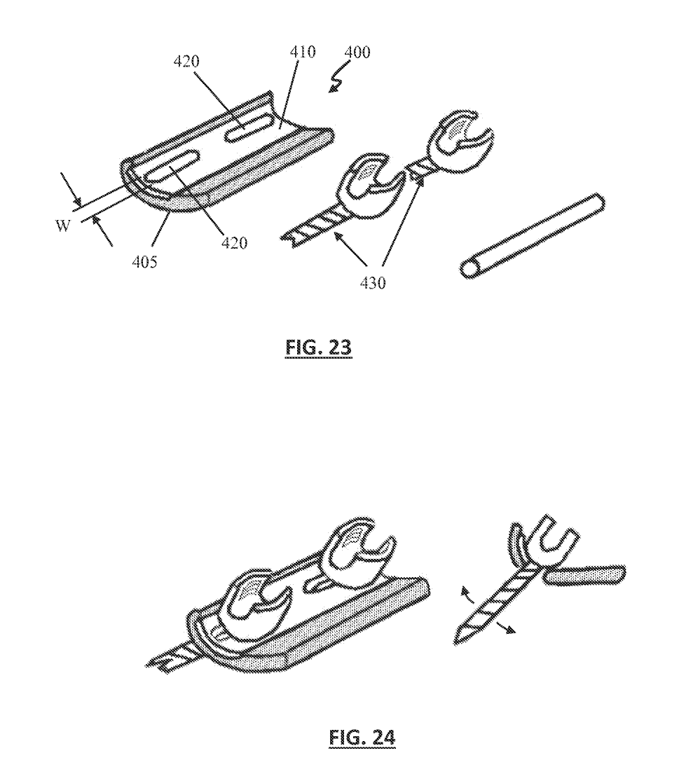

FIGS. 23 through 25 depict another alternative embodiment of an osseointegrative construct that can also be compatible with pre-existing posterior fixation hardware. Although only thoracic, lumbar, and sacral pedicle screws are depicted in the various figures, it should be understood that similar concepts from this embodiment, as well as variants of this invention, could be applied to cervical posterior lateral mass screws, cervical pedicle screws, iliac screws and/or other bony fixation hardware (including hardware located in other areas of the human body).

FIG. 23 shows a composite osseointegrative plate 400. The plate 400 desirably includes an osseointegrative material 405 such as porous or trabecular metal or other material capable of promoting and/or allowing osseointegration, as well as a longitudinally extending support member 410 that could comprise a polymer or metal material (or some other type of sufficiently strong supporting material) based upon the desired rigidity and strength to supplement the osseointegrative material 405. The plate 400 also includes slots 420 through which screw shanks 430 of the depicted standard pedicle screws may be placed. In the depicted embodiment, the medial-lateral dimension (i.e. width W) of the slots dorsally would desirably accommodate the pedicle screw diameter with little extra spacing or gap, whereas ventrally the width of the slits could desirably be wider or enlarged (i.e., a ventral width gap) to allow for angulation of the screw shank (i.e., to accommodate multiple screw trajectories—see FIG. 24). The length of the slots will desirably accommodate multiple inter-screw distances to allow for variations in spine anatomy and screw entry points. One exemplary set of standard poly-axial head pedicle screws and an associated longitudinally extending connecting rod are also depicted in the figure.

FIG. 24 depicts the pedicle screws and osseointegrative plate 400 after the pedicle screw shanks 430 have been advanced through the slots 420 (as previously discussed in connection with FIG. 23). Also depicted is a cross-sectional axial view of the poly-axial pedicle screw-head and plate 400, showing how the ventral width gap can accommodate various possible alignments and/or orientation of the screw shank relative to the plate 400 (see arrows in FIG. 24). Exemplary dorsal and ventral slot width differences can be seen in the figure. The ventral width gap desirably allows the system to accommodate multiple screw trajectories while keeping the screw heads in optimal positions and ensure a secure connection between the plate 400 and the fixation screws.

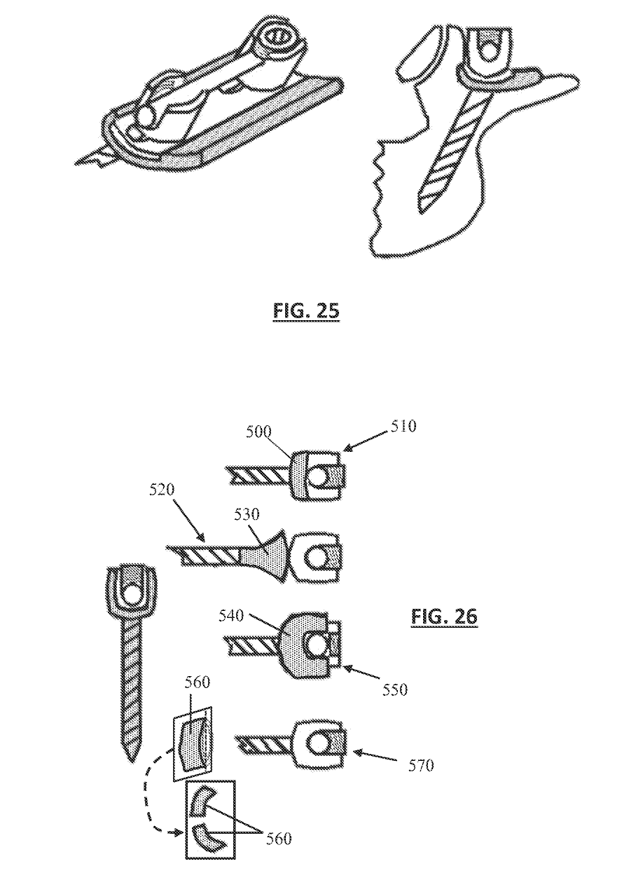

FIG. 25 depicts the pedicle screw and rod assembly of FIG. 24 with a set screw 440 secured into a polyaxial head of the screw. Also shown is a cross-sectional axial in-situ view of the screw and construct 400, showing one example of how the construct 400 orientation can accommodate the pedicle screw's poly-axial head orientation relative to the underlying vertebra.

FIG. 26 depicts additional embodiments constructed in accordance with various of the teachings described herein. In these embodiment, the incorporation of osseointegrative materials, including porous or trabecular metal, into a variety of pedicle screw designs can desirably promote osseointegration between the screws and the underlying bony anatomy. These various embodiment of screw designs could be used independently from the various longitudinal osseointegrative components described herein (i.e., the screws could be used with standard longitudinal rods) as well as in conjunction with longitudinally extending osseointegrative elements described in this invention or variants derived therefrom. In various embodiments, an osseointegrative material path could be formed that completely bridges the vertebral bodies or other bones, while in other embodiments, a partial (incomplete) or multiple complete bridges may be provided.

For example, the bone-facing surface (i.e., bottom) and/or undersurface 500 of a screw's poly-axial head 510 could comprise trabecular or porous metal or other osseoinductive and/or osseoconductive material(s). Alternatively, the proximal portion of a screw 520 could have an integrated and/or removable collar 530 of porous or trabecular metal (or other osseoinductive materials—depicted in shading on the figure) that could be advance into contact with or into a prepared bony surface of the vertebra. As another alternative, a larger collar 540 of porous or trabecular bone (or other osseoinductive materials) could be incorporated with the poly-axial pedicle screw head 550 to allow for contact with the adjacent facets, facet-transverse process junction, and/or the transverse process. As another alternative, a porous or trabecular metal cuff 560 could be used in combination with a standard poly-axial head pedicle screw 570. A cross-sectional view of the cuff 560 is also shown taken along plane CC-CC. In various additional embodiments, most if not all of the surfaces of the pedicle screw and/or poly-axial head exposed to bone could be combined with porous or trabecular metal, or other osseoinductive materials, if desired. Of course, various combinations and variations of these embodiments is also contemplated by the present invention.

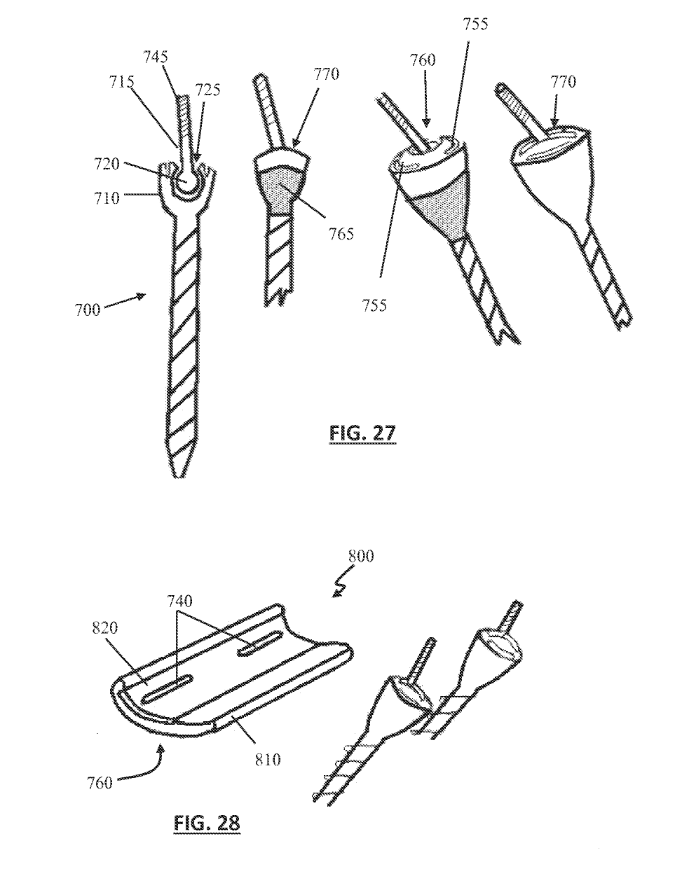

FIGS. 27 through 30 depict another alternative embodiment of an osseoinductive member or linkage constructed in accordance with the various teachings of the present invention. In this embodiment, the member can attach to a specialized pedicle screw that incorporates a poly-axial post. Though only thoracic, lumbar, and sacral pedicle screws are depicted, the same concepts of this embodiment of this invention could be applied to cervical posterior lateral mass screws, cervical pedicle screws, and iliac screws, as well as other spinal; and non-spinal bony fixation devices.

FIG. 27 depicts various alternative design concepts for a pedicle screw incorporating a poly-axial threaded post. The first (left-side) drawing in this figure depicts a cross-sectional view of a pedicle screw 700 having a screw-head 710. Extending outward from the screw-head is a post 715 having a ball-shaped base 720 that sits within and is captured within the head 710 of the pedicle screw. The post 715 extends away from the screw and passes through an opening 725 in the head 710, and can be placed into and through a slotted opening 740 in a longitudinally extending osseointegrative plate, as will be described in conjunction with FIG. 28. The distal end 745 of the post 715 is desirably threaded to accept a locking-nut (not shown) or other securement feature. When the locking nut is tightened down over the threaded post, the ball-shaped base 720 of the poly-axial post will desirably be drawn upward into intimate contact with an inner, upper surface of the screw-head, thereby desirably immobilizing the post relative to the screw and locking into a desired position.

FIG. 27 also shows four (4) notches 755 around the edges of the pedicle screw head 710, which can be employed to accept and accommodate a pedicle screw driver (not shown) or other instruments (of course, varying numbers of such notches could be utilized in a single screw head, including 1, 2, 3, 5, 6 or more notches). A top 770 of the pedicle screw can incorporate a dome-like shape, which in various embodiments may match or conform to a bottom surface 760 of the composite plate (best shown in FIG. 28). The combination of a convex pedicle screw head and a concave bottom surface of the plate can allow for the accommodation of multiple pedicle screw trajectories, and provide a significantly strong linkage between the plate and screw. In various embodiments, the pedicle screw head could include a section 765 that incorporates, is formed from and/or is covered with porous or trabecular metal or other osseoinductive materials. In various embodiments, the hole or opening 725 at the top of the pedicle screw head could be round 760 to accommodate 360 degrees of variability, or it could be elongated 770 to accommodate a broader range of medial lateral screw trajectories. Of course, virtually any shape for the opening 725 could be incorporated into the screw design, if desired.

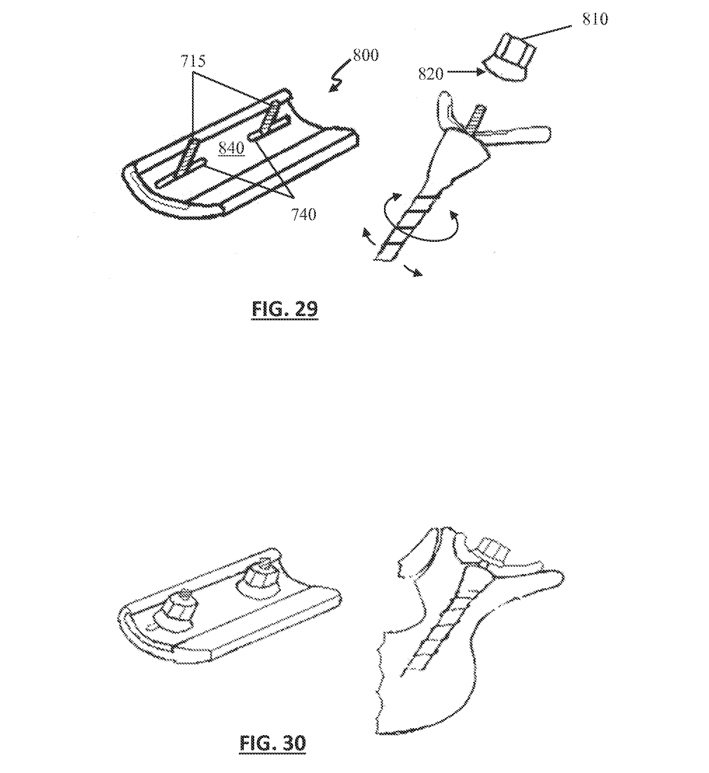

FIG. 28 depicts an embodiment of a composite osseointegrative plate 800 and the associated pedicle screws depicted in FIG. 27. The plate 800 features can include an outer surface 810 having an osseointegrative material such as porous or trabecular metal or other osseoinductive material, along with a longitudinally-extending member or plate 820 comprising a relatively solid material or load-bearing material (i.e., a dense, load-bearing material) that could comprise a wide variety of polymer, ceramics, metals and/or combinations thereof, with the material selection primarily based upon the desired rigidity and strength desired to stabilize the attached bony anatomy until such time as the osseointegrative material facilitates bony ingrowth and the creation of an arthrodesis to stabilize the attached bony anatomy. As previously noted, the plate 800 can also include slots 740 through which the threaded poly-axial posts 715 depicted in FIG. 27 may be placed. The medial-lateral diameter of the slots dorsally will desirably accommodate the pedicle screw diameter with little gap, while ventrally the slots would desirably be wider to accommodate a variety of multiple screw trajectories (as previously described in conjunction with FIG. 24 and as best shown in FIG. 29). In various embodiments, the ventral (i.e. bottom) surface of the plate 800 can include a concave shape (which may include an elongate concave section that extends along the bottom of the plate 800 proximal to the slots 740) which desirably matches the curvature of the convex pedicle screw head. This design could allow for the plate to maintain maximum contact with the pedicle screw heads within a broad range of pedicle screw trajectories. The length of the slots will desirably accommodate multiple interscrew distances to allow for variations in spine anatomy and/or screw entry points.

FIG. 29 depicts the osseointegrative plate 800 and associated pedicle screw posts 715 extending upwards through the slots 740. In this embodiment, the plate 800 has been lowered over the poly-axial posts 715. A cross-sectional view of the plate with polyaxial pedicle screw and post is shown, which highlights the adjustability of the system and the ability of the plate and post design to accommodate a wide variety of positions and possible orientations for the pedicle screw (depicted as arrows in FIG. 29) that the system can accommodate. It should also be noted that the composite plate 800 and the pedicle screw head need not necessarily include an osseoinductive interface such as porous or trabecular metal or other materials (i.e., it may optionally be metal on metal or other solid, non-porous and/or load bearing material(s) for strength purposes). Also shown is a locking nut 810 that can desirably include a convex end or contacting surface 820 where it can contact and interface with an upper surface 840 of the plate 800 when the system is locked. The interface does not necessarily include porous or trabecular metal, but is rather a metal on metal (or equivalent material) for achieving a desired construct strength and durability. As the locking nut 810 is tightened onto the post 715, the polyaxial post 715 is desirably drawn upwards into the plate 800, which locks the post into position (as previously described) and the composite plate 800 is desirably secured to the pedicle screw head and bony anatomy.

FIG. 30 depicts the fully assembled construct of FIG. 29 with the locking nuts in a desired position and secured. An exemplary cross-sectional view of the fully assembled construct is also shown, demonstrating how the pedicle screw can be oriented within the pedicle and vertebral body. It can be seen how the convex screw head mates with and accommodates a multiplicity of orientations relative to the underlying concave portion of the composite plate. In this embodiment, the osseointegrative surface of the composite plate 800 can be in direct apposition and/or contact with the underlying vertebral surfaces (facet, transverse process, and the junction between them), which can include a “bloody bone” surface prepared by the surgeon. The locking nut 810 desirably seats against the concave plate surface, and the top of the poly-axial post 715 is barely visible in FIG. 30.

The examples depicted in the figures above embody various of the potential embodiments of the invention. However, in other broader embodiments, other posterior fixation techniques could be incorporated into the present invention, including the use of clamps, facet screws or screws implanted through other insertion sites and/or trajectories. Furthermore, the various anchoring devices and other means of securing the longitudinal device to the fixation screws can be achieved by a wide variety of connecting mechanisms. The osseointegrative longitudinally-extending device may comprise a single material or a composite of materials. In addition, more durable materials may be chosen based upon the rigidity and strength of fixation desired and/or required. The pedicle screws and set screws described herein may include cannulated designs, which could facilitate minimally invasive, mini-open, or percutaneous implantation.

The advantages of the present invention include, but are not limited to, the ability to provide immediate spinal stability through fixation and, by incorporating osseointegrative materials, longer-term stability similar to those achieved by bony fusion. The present embodiments can achieve such objectives with a single device or combination of devices, such as those depicted and described in the various embodiments of the invention. This invention is versatile and may be used within a wide variety of anatomical areas, including the cervical, thoracic, lumbar, sacral, and iliac spine. By incorporating various composite materials, various degrees of rigidity and strength can be achieved. Further, this invention can accomplish the goals of conventional spinal fusion techniques while minimizing the risks to patients associated with autologous bone harvesting, allograft, and bone graft substitutes.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader, and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. While the foregoing describes specific embodiments, methods, and examples of the present invention, those familiar with the design, manufacture and application of spinal implants may recognize additional concepts that could be combined with the teachings of the present invention, and such combinations, variations, and equivalents to the present invention are fully contemplated herein. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. The invention should therefore not be limited by the specific embodiments, methods, and examples presented herein but by all embodiments and methods within the scope of the invention and the claims that follow.

What is claimed is:

1. A connecting member for stabilizing the spine and maintaining a spacing between at least two anchoring members, the connecting member comprising,
a substantially rigid elongated core having a proximal connection end and a distal connection end, the substantially rigid elongated core comprising a substantially solid, load bearing material;
a substantially porous layer disposed on an outer surface of the elongated core, the substantially porous layer extending from the proximal connection end to the distal connection end;
the proximal and distal connection ends each comprising an opening for accommodating and rigidly connecting to at least one of the at least two anchoring members; and
the at least two anchoring members each comprising a threaded shank for screwing into a vertebrae and an adjustable head, each of the adjustable heads of the at least two anchoring members being sized and configured for securement through at least one of the openings in the proximal and distal connection ends.

2. The connecting member of claim 1, wherein at least one of the openings in the proximal and distal ends comprises a slot that is elongated in a direction substantially parallel to a longitudinal axis of the substantially rigid elongated core.

3. The connecting member of claim 1, wherein each of the openings in the proximal and distal ends comprises a slot that is elongated in a direction substantially parallel to a longitudinal axis of the substantially rigid elongated core.

4. The connecting member of claim 1, wherein a first width of the connection member at a location proximate to the opening in the proximal connection end is greater than a second width of the connection member at a location between the opening in the proximal connection end and the opening in the distal connection end

5. The connecting member of claim 1, wherein each of the at least two anchoring members further comprises an adjustable head that is polyaxially adjustable relative to the threaded shank.

6. The connecting member of claim 1, wherein when the at least two anchoring members are connected to the openings in the proximal and distal connection ends, at least a portion of the at least two anchoring members is in direct contact with the substantially rigid elongated core.

7. The connecting member of claim 1, wherein the porous layer disposed on the outer surface of the elongated core is proximate to the threaded shanks of the at least two anchoring members.

8. The connecting member of claim 1, wherein the substantially porous layer disposed on an outer surface of the elongated core substantially encapsulates the elongated core.

9. The connecting member of claim 1, wherein the substantially porous layer comprises a porous metal.

10. The connecting member of claim 1, wherein the substantially porous layer comprises a trabecular metal.

11. The connecting member of claim 1, wherein the substantially porous layer comprises an osseointegrative material.

12. A medical implant assembly comprising:
a first bone anchor and a second bone anchor, each bone anchor having a bone attachment structure proximal to a first end and a connecting structure on an opposing end;
an elongated connecting member, the elongated connecting member including a first connection region and a second connection region for attachment to the connecting structures of the first and second bone anchors, the elongated connecting member further including a first region comprising substantially solid, non-porous material extending from the first connection region to the second connection region; and
the elongated connecting member further including a substantially porous region extending from the first connection region to the second connection region, the substantially porous region in direct contact with an outer surface of the first region.

13. The medical implant assembly of claim 12, wherein the connecting structure of each of the first and second bone anchors is adjustable relative to the bone anchoring structure of the first and second bone anchors.

14. A device for stabilization of one or more bone segments of the spine, comprising
a first bone anchor assembly for attachment to a first vertebral body of the spine;
a second bone anchor assembly for attachment to a second vertebral body of the spine;
a support structure rigidly connected at a first connection location to the first bone anchor assembly and rigidly connected at a second connection location to the second bone anchor assembly;
the support structure comprising a first region of substantially high strength load bearing material that extends from the first connection location to the second connection location; and
the support structure further comprising a second region of substantially porous, osseointegrative material that extends from the first connection location to the second connection location, at least a portion of the second region in direct contact with a portion of the first region;
wherein when the first and second bone anchor assemblies are rigidly connected to the support structure, at least a portion of the first and second bone anchor assemblies are in direct contact with the first region of substantially high strength load bearing material.

15. The device of claim 14, wherein the first bone anchor comprises a threaded shank for screwing into the first vertebral body of the spine and a head portion, the head portion being polyaxially adjustable relative to the threaded shank.

16. The device of claim 14, wherein the first bone anchor comprises a threaded shank for screwing into the first vertebral body of the spine and a head portion, the head portion being monoaxially adjustable relative to the threaded shank.

17. The device of claim 14, wherein when the first and second bone anchors are rigidly connected to the support structure, at least a portion of the second region of substantially porous, osseointegrative material is in direct contact with both the first and second vertebral bodies.

18. The device of claim 14, wherein the second region of substantially porous, osseointegrative material comprises a metallic material.

19. The device of claim 14, wherein the second region of substantially porous, osseointegrative material comprises a polymer material.

20. The device of claim 14, wherein when the first and second bone anchors are rigidly connected to the support structure and to the first and second vertebral bodies of the spine, at least a first portion of the second region of substantially porous, osseointegrative material is in direct contact with an outer surface of the first vertebral body and at least a second portion of the second region of substantially porous, osseointegrative material is in direct contact with an outer surface of the second vertebral body.
